(19) United States
(12) Patent Application Publication
(10) Pub. No.: US 2007/0081966 A1
(43) Pub. Date: Apr. 12, 2007

(54) Use of alcohols ethoxylated by alpha-hydroxycarboxylic acid esters

(76) Inventors: Ansgar Behler, Bottrop (DE); Hans-Udo Kraechter, Potsdam (DE); Hermann Hensen, Haan (DE)

Correspondence Address: COGNIS CORPORATION;PATENT DEPARTMENT, 300 BROOKSIDE AVENUE, AMBLER, PA, 19002, US

(21) Appl. No.: 10/554,969

(22) PCT Filed: Apr. 21, 2004
(86) PCT No.: PCT/EP04/04208

§ 371 Date: Dec. 15, 2006

(30) Foreign Application Priority Data
Apr. 30, 2003 (DE) 103 19 400.2

(51) Int. Cl. A61K 8/37

(52) U.S. Cl. 424/70.31

(57) ABSTRACT

The disclosed invention relates to the use of alcohols ethoxylated by hydroxy-carboxylic acid esters as moisture dispensers for the skin in cosmetics, especially in preparations containing a surfactant.

### FIELD OF THE INVENTION

This invention relates to the use of hydroxycarboxylic acid esters of ethoxylated alcohols as skin moisturizers in cosmetic and, more particularly, surfactant-containing cosmetic preparations.

### PRIOR ART

Surfactant-containing cosmetic preparations, particularly those containing anionic surfactants, have a strong defatting effect when used frequently, as is typically the case with shower and hair shampoos. To counteract this, moisturizing additives, such as low molecular weight α-hydroxycarboxylic acids, are added to cosmetic preparations. Although these low molecular weight α-hydroxycarboxylic acids, such as glycolic acid or lactic acid, are extremely effective as skin moisturizers, they do cause dermal irritation and have no surfactant properties of their own.

Hydroxycarboxylic acid esters of ethoxylated alcohols are well-known compounds which are used in the form of mono- di- and/or triesters, even in cosmetic preparations. For example, European patent EP 0 852 944 B1 describes the use of citric acid esters for improving the removability of oil-containing cosmetic compositions by washing.

According to European publication EP 0 199 131 A2, citric acid esters are surfactants which may be used in cosmetic preparations, such as hair shampoos.

Nanoemulsions of an oil and alkylether citrates as an anionic surfactant are known in cosmetic preparations from US patent U.S. Pat. No. 6,413,527.

Finally, according to the article by R. Diez et al. in: Proceedings, ₄th World Surfactant Congress, Barcelona (1996), Vol. 2, pp. 129 et seq, alkylether citrates are anionic surfactants which are suitable for cosmetic applications. Citric acid esters of lauryl alcohol with various degrees of ethoxylation (3, 6 and 9), which may be present as mono-, di and/or triesters, were investigated.

The problem addressed by the present invention was to provide compounds for cosmetic preparations, more particularly surfactant-containing cosmetic preparations, which would act as moisturizers without causing any dermal irritation. At the same time, the compounds would themselves have surfactant properties and, in addition, would have other advantages for the skin, such as firming of the skin and wrinkle reduction.

It has surprisingly been found that esters of α-hydroxycarboxylic acids and ethoxylated alcohols act as skin moisturizers without causing any dermal irritation and also have a firming effect on the skin and reduce wrinkles. At the same time, they are anionic surfactants. In addition, it is of particular advantage that the compounds in question have a synergistic foam effect in combination with anionic surfactants, more particularly with ether sulfates.

### DESCRIPTION OF THE INVENTION

The present invention relates to the use of α-hydroxycarboxylic acid esters of ethoxylated alcohols corresponding to formula (I):
R¹O(CH₂CH₂O)ₙH (I)
in which R¹ is a linear or branched alkyl and/or alkenyl group containing 6 to 22 carbon atoms and n is a number of 1 to 50, as moisturizers for the skin in cosmetic preparations.
α-Hydroxycarboxylic Acid Esters

α-Hydroxycarboxylic acids are organic acids which, besides at least one COOH group, contain at least one OH group in the molecule. With one OH group, they may be present as monohydroxycarboxylic acids, with two OH groups as dihydroxycarboxylic acids or with more than two OH groups as polyhydroxycarboxylic acids. Hydroxycarboxylic acids are divided into alpha-, beta- and gamma-hydroxycarboxylic acids according to the position of the OH group to the COOH group.

α-Hydroxycarboxylic acids preferred for the purposes of the invention are tartaric acid, mandelic acid, lactic acid, malic acid, citric acid and salts and self-condensation products thereof. Citric acid is particularly preferred for the purposes of the invention.

The α-hydroxycarboxylic acid esters are derived from ethoxylated C₆₋₂₂ alcohols corresponding to general formula (I):
R¹O(CH₂CH₂O)ₙH (I)
in which R¹ is a linear or branched alkyl and/or alkenyl group containing 6 to 22 carbon atoms and n is a number of 1 to 50. In formula (I), the degree of ethoxylation n is a number of 1 to 20, preferably 1 to 10 and more particularly 3 to 8. Hydroxycarboxylic acid esters derived from ethoxylated alcohols of formula (I), in which R¹ is a linear alkyl group, are particularly suitable.

Typical examples are adducts of on average 1 to 20, preferably 1 to 10 and more particularly 3 to 8 mol ethylene oxide with caproic alcohol, caprylic alcohol, 2-ethylhexyl alcohol, capric alcohol, lauryl alcohol, isotridecyl alcohol, myristyl alcohol, cetyl alcohol, palmitolelyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Adducts of 1 to 10 and more particularly 3 to 8 mol ethylene oxide with technical C₁₂₋₁₈ fatty alcohols, such as for example coconut oil, palm oil, palm kernel oil or tallow fatty alcohol, are preferred.

In one embodiment of the present invention, R¹O in formula (I) is derived from a fatty alcohol mixture containing 65 to 75% by weight C₁₂, 20 to 30% by weight C₁₄, 0 to 5% by weight C₁₆ and 0 to 5% by weight C₁₈ alcohols. This alcohol mixture is commercially available, for example, as Dehydol LS™ from Cognis Deutschland GmbH & Co. KG. Hydroxycarboxylic acid esters based on this fatty alcohol mixture preferably have a degree of ethoxylation n of, on average, 4.

In another embodiment of the present invention, R¹O in formula (I) is derived from a fatty alcohol mixture containing 45 to 60% by weight C₁₂, 15 to 30% by weight C₁₄, 5 to 15% by weight C₁₆ and 8 to 20% by weight C₁₈ alcohols. This alcohol mixture is also commercially available, for example, as Dehydol LT™ from Cognis Deutschland GmbH & Co. KG. Hydroxycarboxylic acid esters based on this selected fatty alcohol mixture have preferably been ethoxylated with, on average, 7 mol ethylene oxide (n =7).

According to the invention, the α-hydroxycarboxylic acids may be completely or, more particularly, partly esterified. In the case of partial esterification, the compounds still contain at least one free carboxyl group. Accordingly, they may be esters or neutralization products thereof. The partial esters are preferably present in the form of the alkali metal, alkaline earth metal, ammonium, alkylammonium, alkanolammonium and/or glucammonium salts.

The most particularly preferred citric acid esters are preferably mixtures of isomeric compounds corresponding to general formula (II): 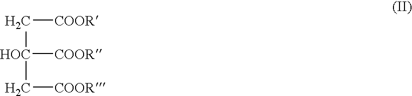
in which R′, R″, R′″ stand for X and/or an ethoxylated alkyl group R¹ with the meaning defined for formula (I), the distribution of the substituents R′, R″ and R′″ having to be such that the ratio by weight of monoester to diester is in the range from 3:1 to 10:1. In a preferred embodiment, the ratio by weight of monoester to diester is in the range from 5:1 to 8:1.

Accordingly, the preferred citric acid ester mixtures according to the invention compulsorily contain mono- and diesters, preferably in quantities of 50 to 90% by weight and more particularly in quantities of 60 to 80% by weight, expressed as mono- and diesters and based on mixture. The mixtures may also contain triesters and free citric acid as the balance to 100% by weight. However, the mixtures preferably contain little free citric acid, preferably less than 10% by weight, based on mixtures.

Accordingly, the preferred citric acid esters according to the invention are mainly partial esters of citric acid which still contain at least one free carboxyl group. The esters may therefore also be acidic esters or neutralization products thereof and X in formula (II) may be hydrogen or a cation. The partial esters are then preferably present in the form of alkali metal, alkaline earth metal, ammonium, alkylammonium, alkanolammonium and/or glucammonium salts (i.e. X=alkali metal, alkaline earth metal, ammonium, alkylammonium, alkanolammonium and/or glucammonium ion).

To produce the citric acid esters preferred for the purposes of the invention, the citric acid must be esterified with the alcohol ethoxylates of formula (I) in a molar ratio of 0.9:1 to 1.1:1 and more particularly 1:1.

The process conditions as such correspond to the prior art. It can be essential to carry out the reaction in a nitrogen atmosphere. In addition, it can be of advantage to adjust a reaction temperature of 150 to 170° C. and preferably 160° C. The citric acid ester mixtures preferred for the purposes of the invention are obtained as the end product. The esters may be present in free form or as salts. In general, a small percentage of the citric acid, preferably less than 10% by weight, remains unesterified for process-related reasons. Reaction products containing at most 8% and, more particularly, at most 5% unesterified citric acid are particularly preferred.

Cosmetic Preparations

According to the invention, the α-hydroxycarboxylic acid esters of ethoxylated alcohols corresponding to formula (I) are used as moisturizers for the skin in cosmetic preparations, more particularly surfactant-containing cosmetic preparations. In the context of the invention, the term “moisturizers” means that these compounds protect the skin against drying out and, at the same time, ensure that the skin retains its natural moisture content.

Through elution of the natural moisturizing factors (NMFs) and washing away of the natural skin lipids after surfactant treatments, the skin undergoes a measurable, temporary drying-out reaction. Mild surfactants and surfactant combinations are normally used to reduce this effect. In the present case, the α-hydroxycarboxylic acid esters of ethoxylated alcohols are not only particularly mild, but also active skin moisturizers. This non-surfactant-like biological mode of action not only exceeds the normal function, it also provides for various new potential applications in formulations or for extended functions.

Accordingly, the compounds used in accordance with the invention may be used for cosmetic preparations such as, for example, hair shampoos, hair lotions, foam baths, shower baths, creams, gels, lotions, alcoholic and aqueous/alcoholic solutions, emulsions, wat/fat compounds, stick preparations, powders or ointments. They also show convincing effects in water-free and water-containing formulations.

The α-hydroxycarboxylic acid esters used in accordance with the invention are preferably used in quantities of 0.01 to 20% by weight and more particularly in quantities of 0.1 to 10% by weight, based on cosmetic preparation.

In one embodiment of the present invention, the cosmetic preparations contain nonionic, anionic, cationic and/or amphoteric/zwitterionic surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, alkyl glucose carboxylates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds and esterquats, more particularly quaternized fatty acid trialkanolamine ester salts. Typical examples of amphoteric or zwitterionic surfactants are alkylbetaines, alkylamidobetaines, amino-propionates, aminoglycinates, imidazolinium betaines and sulfobetaines. Particularly preferred nonionic surfactants are inter alia the alkyl polyglycosides.

According to the invention, the hydroxycarboxylic acid esters are used in cosmetic preparations which preferably contain anionic surfactants, more particularly alkyl and/or alkenyl sulfates and/or alkylether sulfates.

Alkyl and/or alkenyl sulfates, which are often also referred to as fatty alcohol sulfates, are understood to be the sulfation products of primary alcohols which correspond to formula (Ill):
R²O—SO₃M (III)
in which R² is a linear or branched, aliphatic alkyl and/or alkenyl group containing 6 to 22 carbon atoms and preferably 12 to 18 carbon atoms and M is an alkali metal and/or alkaline earth metal, ammonium, alkyl ammonium, alkanolammonium or glucammonium. Typical examples of alkyl sulfates which may be used in accordance with the invention are the sulfation products of caproic alcohol, caprylic alcohol, capric alcohol, 2-ethyl hexyl alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol and the technical mixtures thereof obtained by high-pressure hydrogenation of technical methyl ester fractions or aldehydes from Roelen's oxo synthesis. The sulfation products may advantageously be used in the form of their alkali metal salts and particularly their sodium salts. Alkyl sulfates based on C_{16/18} tallow fatty alcohols or vegetable fatty alcohols of comparable C chain distribution in the form of their sodium salts are particularly preferred.

Alkyl ether sulfates (“ether sulfates”) are known anionic surfactants which, on an industrial scale, are produced by SO₃ or chlorosulfonic acid (CSA) sulfation of fatty alcohol or oxoalcohol polyglycol ethers and subsequent neutralization. Ether sulfates suitable for use in accordance with the invention correspond to formula (IV):
R³O-(CH₂CH₂O)ₘSO₃Z (IV)
in which R³ is a linear or branched alkyl and/or alkenyl group containing 6 to 22 carbon atoms, m is a number of 1 to 10 and Z is an alkali metal and/or alkaline earth metal, ammonium, alkylammonium, alkanolammonium or glucammonium. Typical examples are the sulfates of addition products of on average 1 to 10 and more particularly 1 to 5 mol ethylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof in the form of their sodium and/or magnesium salts. The ether sulfates may have both a conventional homolog distribution and a narrow homolog distribution. It is particularly preferred to use ether sulfates based on adducts of on average 2 to 3 mol ethylene oxide with technical C_{12/14} or C_{12/18} coconut fatty alcohol fractions in the form of their sodium and/or magnesium salts.

The hydroxycarboxylic acids used in accordance with the invention and the surfactants, more particularly alkyl and/or alkenyl sulfates and/or alkylether sulfates, are preferably used in a ratio by weight of 1:20 to 10:1 and more particularly 1:20 to 1:1.

In a most particularly preferred embodiment, the hydroxycarboxylic acid esters used in accordance with the invention are used in cosmetic preparations containing alkylether sulfate as an anionic surfactant. Good synergistic effects in regard to moisture regulation, foaming behavior and dermatological compatibility are observed with such preparations.

The cosmetic preparations may additionally contain oil components, emulsifiers, superfatting agents, pearlizing waxes, consistency factors, thickeners, polymers, silicone compounds, fats, waxes, lecithins, phospholipids, stabilizers, biogenic agents, deodorants, antiperspirants, antidandruff agents, film formers, swelling agents, UV protection factors, antioxidants, hydrotropes, preservatives, insect repellents, self-tanning agents, tyrosine inhibitors (depigmenting agents), solubilizers, perfume oils, dyes and the like as further auxiliaries and additives.

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and tri-glyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched Cr2₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

Suitable emulsifiers are, for example, nonionic surfactants from at least one of the following groups:
products of the addition of 2 to 30 mol ethylene oxide and/or 0 to 5 mol propylene oxide onto linear CB2₂ fatty alcohols, C₁₂-2₂ fatty acids and alkyl phenols containing 8 to 15 carbon atoms in the alkyl group and alkylamines containing 8 to 22 carbon atoms in the alkyl group;
adducts of 1 to 15 mol ethylene oxide with castor oil and/or hydrogenated castor oil;
adducts of 15 to 60 mol ethylene oxide with castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;
partial esters of polyglycerol (average degree of self-condensation 2 to 8), polyethylene glycol (molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;
mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
polyalkylene glycols and
glycerol carbonate.

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layers enhancers for cosmetic formulations from DE 20 24 051 PS.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl4 Isostearate (Isolan® GI 34), Polyglyceryl-3 Oleate, Diisostearoyl Polyglyceryl-3 Diisostearate (Isolan® PDI), Poly-glyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof.

Examples of other suitable polyolesters are the mono-, di- and triesters of trimethylolpropane or pentaerythritol with lauric acid, coconut fatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfac-tants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{2/18} acyl sarcosine.

Finally, cationic surfactants are also suitable emulsifiers, those of the esterquat type, preferably methyl-quaternized difatty acid triethanolamine ester salts, being particularly preferred.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol mono-esters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grunau), quaternized wheat poly-peptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkylene, for example dibromobutane, with bis-dialkylamines, for example bis-dimethylamino -1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycero-phospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Metal salts of fatty acids such as, for example, magnesium, aluminium and/or zinc stearate or ricinoleate may be used as stabilizers.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acid and salts and esters thereof, N-(4-chlorophenyl)-N′-(3,4-dichloro-phenyl) -urea, 2,4,4′-trichloro-2′-hydroxydiphenylether (triclosan), 4-chloro-3,5-dimethylphenol, 2,2′-methylene-bis-(6-bromo-4-chlorophenol), 3-methyl4-(1 -methylethyl)-phenol, 2-benzyl4-chlorophenol, 3-(4-chloro-phenoxy) -propane-1,2-diol, 3-iodo-2-propinyl butyl carbamate, chlor-hexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Dusseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labdanum oil and lavendin oil. The following are preferably used either individually or in the form of mixtures: bergamot oil, dihydromyrcenol, lilial, lyral, citronellol, phenylethyl alcohol, α-hexyl-cinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
nonaqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine.

Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Suitable antidandruff agents are Octopirox® (1-hydroxy4-methyl-6-(2,4,4-trimethylpentyl)-2-(1 H)-pyridinone monoethanolamine salt), Baypival, piroctone olamine, Ketoconazole® (4-acetyl-1-{4-[2-(2,4-dichloro-phenyl) r-2-(1 H-imidazol-1 -ylmethyl)-1,3-dioxylan-c4-ylmethoxyphenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid4-isopropylbenzyl ester, salicylic acid homomenthyl ester;
derivatives of benzophenone, preferably 2-hydroxy4-methoxybenzo-phenone, 2-hydroxy4-methoxy-4′-methylbenzophenone, 2,2′-dihydroxy-4-methoxybenzophenone;
esters of benzalmalonic acid, preferably 4-methoxybenzmalonic acid di-2-ethylhexyl ester;
triazine derivatives such as, for example, 2,4,6-trianilino-(p-carbo-2′-ethyl -1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl) -propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone -5-sulfonic acid and salts thereof;
sulfonic acid derivatives of 3-benzylidene camphor such as, for example, 4-(2-oxo-3-bornylidenemethyl)-benzene sulfonic acid and 2-methyl -5-(2-oxo-3-bornylidene)-sulfonic acid and salts thereof.

Typical UV-A filters are, in particular, derivatives of benzoyl methane such as, for example, 1 -(4′-tert.butylphenyl)-3-(4′-methoxyphenyl)-propane-1,3-dione, 4-tert.butyl-4′-methoxydibenzoyl methane (Parsol 1789) or 1-phenyl -3-(4′-isopropylphenyl)-propane-1,3-dione and enamine compounds. The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa-and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxy-butyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”). Suitable insect repellents are N,N-diethyl-m-toluamide, pentane-1,2-diol or Ethyl Butylacetyl-aminopropionate. A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labdanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

The total percentage content of auxiliaries and additives may be from 1 to 50% by weight and is preferably from 5 to 40% by weight, based on the cosmetic preparation. The preparations may be produced by standard hot or cold processes and are preferably produced by the phase inversion temperature method.

### EXAMPLES

Substances Used

1. Dehydol LT 7™, a product of Cognis Deutschland GmbH & Co. KG, is a fatty alcohol mixture ethoxylated with, on average, 7 mol ethylene oxide. The fatty alcohol mixture has the following chain distribution in % by weight: <C12:0-3%; C12: 48-58%; C14: 18-24%; C16: 8-12%; C18: 11-15%; >C18: 0-1%.

### Example 1

Citric Acid Ester of a C₁₂₋₁₈ Alcohol +7EO; Monoester:Diester 6:1

In a stirred reactor, 28.05 kg (0.146 mol) water-free citric acid and 75.16 kg (0.146 Kmol) Dehydol LT 7™ were heated under nitrogen to 160° C. and stirred at that temperature until the theoretical quantity of water had been released (5.5 hours). A light yellow, clear and liquid product with the following characteristics was obtained:

Characteristics of the citric acid ester of Example 1

| | |
|---|---|
| saponification value | 222 |
| acid value | 132 |
| free citric acid | 2.8% by weight |
| ratio by weight of mono- to diester | 6:1 |

The saponification value (SV) was determined to DGF C-V3.

The acid value (AV) was determined to DIN 53402.

Determination of the Irritation Potential by the RBC Test

The RBC Test was carried out by W. Pape and U. Hoppe's method (Arzneim.-Forsch./Drug Res. 40(1), No. 4 (1990); pp. 498 et seq.)

TABLE 2
RBC Test

| Example | Compound | L/D | Classification |
|---|---|---|---|
| 1 | Citric acid ester of a C₁₂₋₁₈ | >100 | Non-lachrimatory |
| | alcohol + 7EO; mono- to | | |
| | diester ratio: 6:1 | | |

Performance Test: Skin Moisture
Background

The epidermis of human skin includes the horny layer (the Stratum corneum). The Stratum corneum is a dielectric medium of low electrical conductivity. The water content leads to increased dielectrical conductivity, so that determination of the dielectrical conductivity of the Stratum corneum can be used as a measure of the moisture content of human skin. Any increase in the dielectrical conductivity of the Stratum corneum reflects an increase in the moisture content of human skin. The dielectrical conductivity of the Stratum corneum was determined with a skin surface hygrometer (SKICON 200, IBS CO., Hamamatsu, Japan) at 3.5 MHz.

Method

Samples of normal skin obtained from plastic surgery were used for this test. The Stratum corneum from these skin samples was stored in chambers with defined relative humidity (44%, saturated potassium carbonate solution) and standardized. Each sample of the Stratum corneum was comparatively tested under five conditions:
1. no treatment;
2. placebo treatment;
3. treatment with a preparation consisting of a hydrogel (Hydrogel LS from Laboratoire Serobiologique LS) containing 1% w/v of the above-described moisturizing citric acid ester;
4. treatment with a preparation consisting of a hydrogel (Hydrogel LS from Laboratoire Serobiologique LS) containing 2% w/v of the above-described moisturizing citric acid ester;
5. treatment with a preparation consisting of a hydrogel (Hydrogel LS from Laboratoire Serobiologique LS) containing 5% w/v of the above-described moisturizing citric acid ester.

The placebo was the hydrogel (Hydrogel LS from Laboratoire Serobiologique LS) without the moisturizing citric acid ester described above.

The treatment with the preparations was carried out 3 times at 30-minute intervals. The quantity applied was 1 mg/cm². The conductivity measurement was carried out before the treatment and then up to 24 hours after the third treatment. Table 2 below shows the moisturizing effect as determined by measurement of the dielectrical conductivity (in μS) of the moisturizing citric acid ester described above; mean value of 10 measurements (the standard deviation is shown in brackets).

A dose-dependent moisture-regulating activity is apparent from the results.

TABLE 2
Moisturizing effect in μS

| Type of | Before | | | | | | |
|---|---|---|---|---|---|---|---|
| treatment | treatment | 30 Mins. | 1 Hour | 2 Hours | 4 Hours | 6 Hours | 24 Hours |
| Control | 15.9 (1.8) | 22.3 (2.0) | 22.4 (1.7) | 21.1 (1.7) | 20.8 (1.3) | 19.7 (1.9) | 18.0 (1.4) |
| Placebo | 15.9 (0.9) | 38.8 (2.4) | 31.9 (2.8) | 27.7 (1.8) | 27.6 (2.9) | 26.5 (3.0) | 24.8 (2.2) |
| Treatment 3. | 16.2 (1.2) | 55.2 (5.4) | 46.1 (4.6) | 36.4 (4.6) | 37.9 (3.9) | 32.0 (3.5) | 32.2 (4.4 |
| Treatment 4. | 16.8 (1.4) | 86.1 (9.3) | 63.1 (3.4) | 52.5 (4.1) | 47.3 (2.7) | 42.6 (2.9) | 38.6 (2.8) |
| Treatment 5. | 17.3 (1.6) | 113.0 (8.0) | 82.7 (5.4) | 69.5 (4.3) | 54.9 (2.5) | 48.2 (2.6) | 47.4 (3.5) |

TABLE 3
Percentage increase in moisture compared with placebo (in %)

| Type of | Before the | | | | | | |
|---|---|---|---|---|---|---|---|
| treatment | treatment | 30 Mins. | 1 Hour | 2 Hours | 4 Hours | 6 Hours | 24 Hours |
| Treatment 3. | 1 | 42 | 45 | 31 | 37 | 21 | 30 |
| Treatment 4. | 5 | 122 | 98 | 89 | 71 | 61 | 56 |
| Treatment 5. | 9 | 192 | 159 | 150 | 99 | 82 | 91 |

Measurement of the moisture level shows that the citric acid ester tested clearly has a very good and significant effect.

1-11. (canceled)

12. A process for improving the moisturizing effect of a surfactant-containing cosmetic composition, comprising combining one or more α-hydroxycarboxylic acid esters of ethoxylated alcohols of formula (I):
R¹O(CH₂CH₂O)ₙH (1)
in which R¹ is a linear or branched alkyl or alkenyl group containing 6 to 22 carbon atoms and n is a number from 1 to 50, with the surfactant-containing cosmetic composition.

13. The process according to claim 12 wherein, in formula (I), n is a number from 1 to 10.

14. The process according to claim 12 wherein, in formula (I), R¹ is a linear alkyl group.

15. The process according to claim 12 wherein the α-hydroxycarboxylic acids are selected from the group consisting of tartaric, mandelic, lactic, malic and citric acids and salts and self-condensation products thereof.

16. The process according to claim 12 wherein the α-hydroxycarboxylic acid is partially esterified so that it contains one free carboxyl group which may be present in the form of the alkali metal, alkaline earth metal, ammonium, alkylammonium, alkanolammonium or glucammonium salt.

17. The process according to claim 16 wherein the α-hydroxycarboxylic acid is citric acid.

18. The process according to claim 12 wherein, in formula (I), R¹CO is derived from a fatty alcohol mixture containing 65 to 75% by weight C₁₂, 20 to 30% by weight C₁₄, 0 to 5% by weight C₁₆ and 0 to 5% by weight C₁₈ alcohol.

19. The process according to claim 18 wherein, in formula (I), n has a mean value of 4.

20. The process according to claim 12 wherein, in formula (I), R¹CO is derived from a fatty alcohol mixture containing 45 to 60% by weight C₁₂, 15 to 30% by weight C₁₄, 5 to 15% by weight C₁₆ and 8 to 20% by weight C₁₈ alcohol.

21. The process according to claim 20 wherein, in formula (I), n has a mean value of 7.

22. The process according to claim 12 wherein the surfactant in the surfactant-containing cosmetic composition comprises an anionic surfactant.

23. The process according to claim 12 wherein the surfactant in the surfactant-containing cosmetic composition is selected from one or more of the group consisting of alkyl sulfates, alkenyl sulfates and alkylether sulfates.

24. The process according to claim 12 wherein the ratio of α-hydroxycarboxylic acid ester to surfactant, by weight, is 1:20 to 10:1.

25. A skin moisturizer composition, comprising
(A) one or more α-hydroxycarboxylic acid esters of ethoxylated alcohols of formula (I):
R¹O(CH₂CH₂O)ₙH (I)
in which R¹ is a linear or branched alkyl or alkenyl group containing 1 to 10 carbon atoms and n is a number from 1 to 50, wherein, in formula (I), R¹CO is derived from
(i) a fatty alcohol mixture containing 65 to 75% by weight C₁₂, 20 to 30% by weight C₁₄, 0 to 5% by weight C₁₆ and 0 to 5% by weight C₁₈ alcohol, or
(ii) a fatty alcohol mixture containing 45 to 60% by weight C₁₂, 15 to 30% by weight C₁₄, 5 to 15% by weight C₁₆ and 8 to 20% by weight C₁₈ alcohol, and wherein the α-hydroxycarboxylic acids are selected from the group consisting of tartaric, mandelic, lactic, malic and citric acids and salts and self-condensation products thereof; and
(B) an anionic surfactant.

26. The cosmetic composition according to claim 25 wherein, in formula (I), n is a number from 3 to 8.

27. The cosmetic composition according to claim 25 wherein, in formula (I), R¹ is a linear alkyl group.

28. The cosmetic composition according to claim 25 wherein the α-hydroxycarboxylic acid is partially esterified so that it contains one free carboxyl group which may be present in the form of the alkali metal, alkaline earth metal, ammonium, alkylammonium, alkanolammonium or glucammonium salt.

29. The cosmetic composition according to claim 28 wherein the α-hydroxycarboxylic acid is citric acid.

30. The cosmetic composition according to claim 25 wherein the anionic surfactant is selected from one or more of the group consisting of alkyl sulfates, alkenyl sulfates and alkylether sulfates.
